(19) United States
(12) Patent Application Publication
(10) Pub. No.: US 2009/0095675 A1
(43) Pub. Date: Apr. 16, 2009

(54) Water treatment bioreactor using hollow filaments

(75) Inventors: Antonius J. Runneboom, Tokyo (JP); Reid Bowman, Ojai, CA (US); David D. Friese, Orinda, CA (US); Ryan S. Overstreet, Discovery Bay, CA (US); Dennis P. Smith, Clayton, CA (US)

Correspondence Address: King & Spalding LLP, P.O. Box 889, Belmont, CA, 94002-0889, US

(73) Assignee: Applied Process Technology, Pleasant Hill, CA (US)

(21) Appl. No.: 12/283,745

(22) Filed: Sep. 12, 2008

Related U.S. Application Data
(60) Provisional application No. 60/993,964, filed on Sep. 13, 2007.

(51) Int. Cl. C02F 3/00; B32B 1/08; B32B 5/02; C02F 103/00; C12M 3/00

(52) U.S. Cl. 210/615; 428/36.9; 442/334; 435/289.1

(57) ABSTRACT

Apparatus, systems, and methods for removal of contaminants in water are described. In particular, apparatus, systems, and methods for the treatment of potable water, for removal of oxidized contaminants, using hollow polyester filaments for sustaining a biofilm are described.

### CROSS-REFERENCE TO RELATED APPLICATIONS

This application claims the benefit of U.S. Provisional Application No. 60/993,964, filed Sep. 13, 2007, incorporated herein by reference in its entirety.

### TECHNICAL FIELD

The apparatus, systems, and methods for use in water treatment are described. More particularly, the apparatus, systems, and methods are directed to treatment of water contaminated with one or more contaminants. In preferred embodiments, the apparatus, systems, and methods are for the treatment of potable water, for removal of, for example, oxidized contaminants or reduced contaminants.

### BACKGROUND

Industries are continuously laboring toward the goal of removing pollutants from contaminated water to make the water safe and usable, at both the ground level and the consumer level. Government-regulated agencies establish limits for many common industrial pollutants. These limits tend to become stricter as pollution reduction and removal technology proves effective at accomplishing previously-established requirements. Consequently, both ground and consumer level water continue to improve in terms of both purity and safety.

One method to reduce or remove pollutants is bioremediation. In a broad sense, bioremediation includes the use of microorganisms that digest pollutants as a source of food, including nitrogen and carbon compounds. Bacterial metabolism converts the pollutants to metabolites having a simple chemical structure, sometimes degrading the pollutants completely to carbon dioxide and water in an aerobic process, or to methane in an anaerobic process. The metabolites produced by the bacteria typically have no adverse environmental effects.

Municipal, agricultural, and industrial brines and other waste waters are often treated to remove contaminants before reuse or return of the waste water to the environment. Contaminants that are typically found in waste waters include nitrate (NO₃⁻), nitrite (NO₂⁻), ammonia, and other nitrogenous compounds, and perchlorate (ClO₄⁻). Ammonia is widely used in the manufacture of fertilizers, rocket fuels, and myriad other chemicals, and ammonia pollution is frequently caused by over-fertilization, intensive livestock farming, and human waste. Elevated concentrations of ammonia or nitrate in waters spur eutrophication, which can lead to hypoxia, odors, color, and other undesirable water-quality changes. In addition, ammonia is directly toxic to fish and exhibits a large oxygen demand, which affects many organisms.

Nitrite is a nitrogenous contaminant associated with methemoglobinemia and other forms of cancer. Nitrate is converted to nitrite in the human digestive system. The U.S. Environmental Protection Agency (EPA) has set a maximum contaminant level (MCL) of 10 mg NO₃⁻-N/L and 1 mg NO₂⁻-N/L for drinking water. Standards for release of wastewater into the environment are more stringent, and commonly require a total nitrogen level in the wastewater of between about 1-3 mg total nitrogen per Liter (total-N/L).

Perchlorate is an oxidized anion that can originate from various salts. Ammonium perchlorate is an ingredient of solid rocket fuel, highway safety flares, fireworks, matches, and air bag inflators. Because perchlorate may inhibit thyroid function, its concentration in drinking water is subject to governmental regulations. For example, the state of California recently lowered its perchlorate drinking water acceptance level from 18 μg/L to 4 μg/L. A recent EPA study suggested 1 μg/L as a treatment goal for drinking water. Perchlorate can be removed by perchlorate-reducing bacteria, which use perchlorate as an electron acceptor in their metabolism. Under anaerobic conditions, such bacteria typically produce chloride and oxygen.

Denitrification is a process of reducing nitrite and nitrate to nitrogen gas (N₂). Because drinking water typically has low concentrations of biodegradable organic materials and is, therefore, “oligotrophic,” reduction of nitrite and nitrate typically requires addition of an organic (heterotrophic denitrification) or inorganic (autotrophic denitrification) electron (e⁻) donor. In heterotrophic denitrification, ethanol, methanol, and acetate are common e⁻ donor substrates for drinking water. Hetertrophic denitrification of drinking water has several disadvantages, which originate from the after-process residuals due to, e.g., overdosing or variation of influent nitrate concentration. The presence of residual organic electron donors in the drinking water creates biological instability. The residual effect can further be problematic if the electron donor is harmful. Denitrification using hydrogen (H₂) gas as the electron-donor substrate is called autohydrogenotrophic denitrification. The H₂-oxidizing reaction is performed by autotrophs (i.e., bacteria that use an inorganic carbon source). In this process, H₂ gas is introduced into the system, for example by sparging, which results in the near saturation of waste water with dissolved H₂ (1.6 mg/L at 20° C.).

In contrast to denitrification, nitrification is a process of removing reduced contaminants from water. Nitrification is illustrated by, for example, reduction or removal of ammonium species (NH₄⁺) in waste water to nitrate (e.g, NO₃⁻) by nitrifying bacteria in the presence of oxygen (NH₄⁺2O₂→NO₃⁻2H⁺+H₂O).

The present apparatus, systems, and methods relate to an apparatus, systems, and methods for removing contaminants from water by means of denitrification and/or nitrification.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, an apparatus is provided, the apparatus comprising a plurality of hollow filaments, each hollow filament comprised of a polyester.

In some embodiments, each hollow filament in the plurality of hollow filaments is comprised of a polyester selected from the group consisting of poly(ethylene terephthalate) (PET), poly(trimethylene terephthalate) (PTT), poly(butylene terephthalate) (PBT), poly(ethylene naphthalate) (PEN), poly(cyclohexylene dimethylene terephthalate) (PCTA), poly(butylene naphthalate) (PBN), polycarbonate (PC), and poly(lactic acid) (PLA). In particular embodiments, the polyester is poly(ethylene terephthalate) (PET).

In some embodiments, each hollow filament in the plurality of hollow filaments is comprised of a polyester having a carboxylate ester of the form R₁—C(═O)OR₂ in its monomeric repeat unit.

In some embodiments, the plurality of hollow filaments is secured at one end to an end cap or to a tube sheet.

In another embodiment, the plurality of hollow filaments form a fabric. In still another embodiment, the plurality of hollow filaments are sealable at one end and open at an opposite end, and the fabric is secured to a center tube.

In yet another embodiment, the plurality of hollow filaments are in the form of a fabric and a spacer material is adjacent the fabric. In some embodiments, a plurality of fabric layers is alternated with a plurality of layers of a spacer material.

In another aspect, an apparatus comprising a plurality of hollow filaments is provided, each hollow filament comprised of a material selected from the group consisting of a polyester, a polyamide, a halogenated polymer, a non-halogenated polyolefin, and a sulfur-containing polymer. In particular embodiments, the non-halogenated polyolefin is for example, polypropylene and the halogenated polymer is polyvinylchloride.

In one embodiment, the hollow filaments comprised of such a material is a fabric. In other embodiments, the plurality of hollow filaments are in the form of a fabric and a spacer material is adjacent the fabric. In some embodiments, a plurality of fabric layers is alternated with a plurality of layers of a spacer material.

In another aspect, a bioreactor is provided, the bioreactor comprising a plurality of hollow filaments, a casing having a cavity in which the plurality of hollow filaments are received, and a port in the bioreactor.

In one embodiment, the port in the bioreactor is in fluid communication with the lumens of the plurality of hollow filaments, and can be used, for example, for introducing a gas into the lumen of the filaments. In another embodiment, the port in the bioreactor is in fluid communication with the external surfaces of the filaments, and can be used, for example, to introduce a nutrient or chemical to the water being treated in the bioreactor.

In another embodiment, the casing in the bioreactor includes a drain valve that is movable between open and closed positions, and when in a closed position closes one end of the plurality of hollow filaments such that the lumens of the hollow filaments are sealed in a fluid tight manner from communication with the external environment.

In some embodiments, the bioreactor is situated downstream from a denitrification process supplied with a carbon source. In some embodiments, the carbon source is selected from methanol, a solution containing methanol, acetate, propionate, isobutyrate, butyrate, valerate, malate, fumerate, lactate, chlorate, catechol, glycerol, and citrate.

In a related aspect, a method of treating wastewater is provided, comprising providing a plurality of hollow filaments, the hollow filaments each having an exterior surface and an interior surface defining a hollow interior, and being comprised of a polyester; providing a gas to the hollow interiors of the hollow filaments, the gas being permeable through the fibers, and contacting wastewater with the exterior surfaces of the hollow filaments, wherein a biofilm on the exterior surfaces of the plurality of hollow filaments removes or reduces the level of a contaminant in the wastewater.

In some embodiments, the gas is hydrogen. In other embodiments, the gas is oxygen, nitrogen, or a mixture of gases, selected from at least two gases selected from air, hydrogen, carbon dioxide, oxygen, and nitrogen.

In another embodiment, a disinfectant is provided to the wastewater treatment system. The disinfectant in some embodiments is introduced to the treated (decontaminated) water after it has been treated in a bioreactor. In one embodiment, the disinfectant is a gas, such as chlorine dioxide or chlorine gas.

In some embodiments, the polyester used in the method is selected from the group consisting of poly(ethylene terephthalate) (PET), poly(trimethylene terephthalate) (PTT), poly(butylene terephthalate) (PBT), poly(ethylene naphthalate) (PEN), poly(cyclohexylene dimethylene terephthalate) (PCTA), polycarbonate (PC), poly(butylene naphthalate) (PBN), and poly(lactic acid) (PLA).

Additional embodiments of the present disclosure will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present apparatus, systems, methods, and compositions are set forth in the following description and claims, particularly when considered in conjunction with the accompanying examples and drawings.

### BRIEF DESCRIPTION OF THE DRAWINGS

FIG. 1 is a schematic of an exemplary bioreactor system utilizing hollow polyester filaments.

FIG. 2A is a schematic of another exemplary bioreactor containing hollow filaments.

FIG. 2B is a photograph showing a prototype bioreactor containing hollow filaments.

FIG. 3 is a schematic of an exemplary water treatment system comprising two membrane biofilm reactors (MBfR) comprising modules with hollow fiber membranes (filaments).

FIG. 4A is a schematic of an exemplary water treatment system for use in municipal water treatment, the system comprising two bioreactors comprising hollow polyester filaments, along with aeration, filtration and disinfection components.

FIG. 4B is a schematic of another exemplary water treatment system comprising two bioreactors, along with a pH control component, an element for mixing or aerating the biofilm, and aeration, filtration and disinfection components.

FIG. 5A is a plane, side view of a bioreactor.

FIG. 5B is a cross-sectional view of one end of the module in the bioreactor of FIG. 5A.

FIGS. 6A-6B are illustrations of a fabric comprising polyester hollow filaments (FIG. 6B), and a braid of polyester hollow filaments (FIG. 6B).

FIG. 7A is a graph of nitrate removal, in percent, as a function of nitrate loading, in mass of nitrate per surface area of the hollow filaments per day (mg-N/m²-day), during treatment of wastewater in a system comprising a single bioreactor having one module with polyester hollow filaments, the bioreactor operated with a recycle rate of 10 gallons per minute (0.63 L/s, open circles) or 2 gallons per minute (0.13 L/s, closed circles).

FIG. 7B is a graph of total nitrogen removal, in percent, as a function of nitrate loading rate, in mg-N/m²-day, in a system for treatment of wastewater, the system comprising a single bioreactor module with polyester hollow filaments, the bioreactor operated with a recycle rate of 10 gallons per minute (0.63 L/s, open circles) or 2 gallons per minute (0.13 L/s, closed circles).

FIG. 7C is a graph of nitrate flux, in mg-N/m²-day, as a function of nitrate loading rate, in mg-N/m²-day, in a system for treatment of wastewater, the system comprising a single bioreactor module with polyester hollow filaments, the bioreactor operated with recycle rates of 10 gallons per minute (0.63 L/s, open circles) or 2 gallons per minute (0.13 L/s, closed circles).

### DETAILED DESCRIPTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art.

All publications, patents and patent applications cited herein, whether supra or infra, are hereby incorporated by reference in their entirety.

It must be noted that, as used in this specification, the singular forms “a,” “an,” and “the” include plural referents unless the context clearly dictates otherwise.

The apparatus, systems, and methods are directed to an autohydrogenotrophic reactor for removal of oxidized contaminants from water. Oxidized contaminants include but are not limited to nitrate, nitrite, perchlorate, chlorate, bromate, selenate, hexavalent chromium, arsenate, and a range of chlorinated contaminants, etc. A feature of the present autohydrogenotrophic reactor is the use of polyester in fabricating the plurality of hollow filaments of the bioreactor. Polyester offers a number of advantages in terms of cost, recycling, and disposal. The apparatus, systems, and methods are also directed to a bioreactor for removal of reduced contaminants, such as ammonia, from water. Accordingly, the present apparatus, system, and methods are suitable for decontaminating waste water or ground water to produce potable water for agricultural or industrial use, or even drinking water.

### II. Polyester Filaments

In one embodiment, the apparatus and systems described herein comprise a plurality of hollow filaments. The hollow filaments can be formed from a variety of materials, including a polyester, a polyamide, a halogenated polymer (such as polyvinylchloride), a non-halogenated polyolefin (such as polypropylene), and a sulfur-containing polymer. In one embodiment, the material is not polymethylpentene or a polyurethane. In a preferred embodiment, the hollow filaments are comprised of a polyester. Esters are a class of organic compounds traditionally formed by the condensation of an alcohol and an organic acid. Where the acid is a carboxylic acid, the resulting ester has the structure R1-C(═O)OR2, where R1 can be hydrogen or selected from a myriad of functional groups, which will be apparent to a skilled artisan based on the polyesters noted below, and R2 is one of myriad functional groups apparent to a skilled artisan based on the polyesters noted below. In a preferred embodiment, R1 is a terephthalate group and R2 is an ethylene group, to form poly(ethylene terephthalate). In another exemplary embodiment, R1 is a naphtalalate group and R2 is an ethylene group, to form poly(ethylene naphthalate). Other examples of R1 and R2 are evident from the exemplary polyesters noted below.

Esters can also be formed from phosphoric, sulfuric, nitric, boric, benzoic, and other acids. Cyclic esters are known as lactones. Esters participate in hydrogen bonding as hydrogen-bond acceptors. However, esters do not function as hydrogen donors. This allows esters groups to form hydrogen bonds with many other functional groups, while precluding hydrogen-bonding between esters groups. Esters are generally hydrophobic, although the nature of the R1 and R2-groups affects the characteristics of a particular ester.

Polyester is a polymer of one or more preselected ester monomers, typically produced by azeotrope esterification, alcoholic transesterification, acylation (i.e., the HCl method), the silyl or silyl acetate method, or the ring-opening method, and variations, thereof, depending on the particular polyester. Polyester is widely used in the manufacture of consumer products, and its mechanical properties are well known.

Polyesters include but are not limited to poly(ethylene terephthalate) (PET), poly(trimethylene terephthalate) (PTT), poly(butylene terephthalate) (PBT), poly(ethylene naphthalate) (PEN), poly(cyclohexylene dimethylene terephthalate) (PCTA), polycarbonate (PC), poly(butylene naphthalate) (PBN), and poly(lactic acid) (PLA). Polyesters may be homopolymer or heteropolymers. As used herein, heteropolymers include copolymers. A common polyester co-polymers is 1,4-cyclohexanedimethanol (CHDM). For example, PCTA is a copolymer of three monomers, which are terephthalic acid, isophthalic acid, and CHDM. While some industries use the terms “polyester” and “PET” almost interchangeably, the term “polyester” refers to the entire class of compounds.

Many of the advantages of polyester are most apparent when fibers are tossed or woven into tows, ropes, fabrics, etc. For example, polyester is widely used in the textile industry. The most widely used polyester is PET (or PETE), which exists in amorphous (transparent) and semi-crystalline (white or opaque) forms and is readily made into fibers and textile sheets.

In addition to being inexpensive to produce, polyesters are particularly strong, resilient, resistant to abrasion, and resistant to stretching and shrinking. Polyester textiles are wrinkle resistant, mildew resistant, fast drying, and retain heat-set pleats and creases. Polyester displays excellent resistance to oxidizing agents, cleaning solvents, and surfactants. While resistant to sunlight, UV stabilizers are typically added for use outdoors or exposed to UV light.

Polyesters, like most thermoplastics, are recyclable and may be may be virgin polyesters, recycled polyesters, post consumer polyesters, recycled monomers, or combinations and variations, thereof. Some polyesters, including PET, offer the additional advantage of containing only carbon, oxygen, and hydrogen (i.e., no sulfur, phosphorus, nitrogen, etc.), which makes them candidates for incineration.

Exemplary polyester hollow filaments are made of melt-spinnable polyester, such as PET, that is melted and pressed through a hole of a spinneret, quenched in water or in an air stream, stretched in one or more steps in combination with heating, and then wound onto on a spool using a winding machine. The hollow filaments are fine, effectively “endless” flexible hollow polyester tubes, which can be cut to any length as needed. The filaments having an exterior surface that is typically exposed to the wastewater, and an interior surface for interacting with sparged gas. The interior surface defines a hollow interior space. The preferred diameter of polymer filaments depends on the particular embodiment. The polyester filaments may be less than 500 μm in diameter, or even less than 100 μm in diameter. The filaments, may even be less than 50 μm, less than 20 μm, or even less that 10 μm in diameter. The hollow filaments may have a uniform diameter or be heterogenous with respect to diameter. Where the filaments are of heterogenous diameter, the diameter may fall within a preselected range.

The hollow polyester filaments may be tossed into bundles to form multifilament yarns, which are then assembled into modules for use in a bioreactor, to be described. Filaments of less than 10 dtex (i.e., decitex=1 gram per 10,000 meters) are preferred for yarns, while filaments of more than 100 dtex are typically used as monofilaments. Intermediate filaments are used in either form. Both mono and multifilaments can be used as warp or weft in technical fabrics. Loose bundles of filament that are not woven into fabrics may also be used.

The diffusion of gas through a polymer membrane is generally described by Fick's laws of diffusion. The solubility coefficient depends on the particular polymer-gas combination and Henry's law. The permeation of low molecular weight gases in rubbery polymers (below their glass transition temperatures) at moderate pressures is Fickian and follows Henry's law for different sorption modes (i.e. absorption, the adsorption, plus trapping in microvoids, clustering, and aggregation). Klopffer, M. H. and Flaconnèche, B., Oil & Gas Science and Technology—Rev. IFP, 56, 2001, No. 3).

The burst pressure of a hollow polyester filament can be calculated using the formula:

P=T·(OD²−ID²)/(OD²+ID²)

where P is burst pressure, T is tenacity, and OD and ID are outside and inside diameter, respectively. OD and ID are preselected variables and tenacity is a constant associate with a particular polymer.

For example, a PET filament having and OD of 200 μm, an ID of 100 μm, and a tenacity of approximately 30 cN/tex (i.e., 400 MPa) has a burst pressure of 240 MPa. A working pressure of about 1% of the burst pressure is, therefore, 2.4 MPa, or 337 psi. In one embodiment, an apparatus comprised of a plurality of filaments is provided, where the filaments have a burst pressure P, and the apparatus is operated at a pressure of less than about 50% of the burst pressure P, more preferably of less than about 30% of the burst pressure P, still more preferably of less than about 25% of the burst pressure P, even more preferably at less than about 10% of the burst pressure P, and still more preferably at less than about 5% or less than about 2% of the burst pressure P. By way of example, an apparatus or system comprising a PET filament with a burst pressure P of 337 psi is preferably operated at a pressure of about 168.5 psig (50% of the burst pressure) or at a pressure of about 84.2 psig (25% of the burst pressure), and so on.

Preferred filament diameters for use as described are from, in one embodiment about 50 μm to about 5000 μm, or, in another embodiment, from about 100 μm to 3000 μm. The optimal shape of hollow filaments is round, although irregular shaper are expected to produce satisfactory results. Consistent density is preferred but not required. Preferred tenacity values are from about 10 to about 80 cN/tex, or even from about 20 to about 60 cN/tex.

Percent void volume (% V) may be calculated using the formula:

% V=(inside cross-sectional area)/(outside cross-sectional area)×100

An acceptable range for void volume is from about 1% to about 99%, while a preferred range for some embodiments is from about 25% to about 50%.

### III. Bioreactor Apparatus and System

In one aspect, a bioreactor apparatus and a system comprising at least one bioreactor is provided, for use in reducing the level of or removing contaminants from a waste stream or from a body of water. In one embodiment, an apparatus comprised of a plurality of hollow filaments is provided, and depending on the intended use, the hollow filaments can be comprised of, for example, a polyester. The apparatus in one embodiment is a bioreactor that comprises the plurality of hollow filaments and an inlet port for introducing a gas into the lumen of the hollow filaments. In another embodiment, the apparatus is a bioreactor comprised of a plurality of hollow filaments, a casing having a cavity in which the plurality of hollow filaments is disposed, and a port in the casing. In some embodiments, the casing includes at least two ports, one in fluid communication with the lumens of the plurality of hollow filaments, and a second port in fluid communication with the external surfaces of the plurality of hollow filaments. In another embodiment, the apparatus includes a drain valve, as will be described below.

During use in removing or reducing the level of contaminants from waste water, a biofilm is established on the external surfaces of the filaments. Gas introduced into the lumen of the hollow filaments diffuses through the wall of the filaments, becoming available to a biofilm growing on the outside of the filaments. The microorganisms in the biofilm facilitate removal of the contaminants, where as described in more detail below, nitrification bacteria utilize oxygen to facilitate oxidation of a reduced nitrogen component (e.g., ammonium or ammonia) to, for example, nitrate, and denitrification bacterial utilize hydrogen to reduce an oxidized contaminant to nitrogen gas. In other embodiments, the bioreactor additional includes and an external casing that contains the hollow filaments, and the casing has at least an exit port for discharge of partially treated or treated water. In still another embodiment, the bioreactor includes a drain in fluid communication with the lumen of the filaments. The drain is movable between an open position and a closed position. When in the open position, components that may accumulate within the filaments' lumens, such as water or components present in the gas introduced into the lumens of the filaments, such as inert gases, that do not permeate across the filaments to the external water side of the bioreactor, can be released or removed. The drain, when in the closed position, provides a fluid tight seal of the hollow filament lumens at the end at which the drain is positioned.

As mentioned above, in a preferred embodiment, the hollow filaments are comprised of a polyester. The use of polyester hollow filaments offers several advantages over other polymers and materials. First, the extent of biofilm loss due to abrasion and “washing away” is much less in the hollow-fiber biofilm reactor (herein “bioreactor”) than in the fluidized-bed bioreactor. The ester groups of polyester are cable of forming interactions with bacteria, improving biofilm accumulation and stability.

Second, since the biofilm is on the surface of the hollow filaments, the gas introduced into the lumen of the filaments, typically hydrogen or oxygen, permeates the wall of the filament and directly contacts the biofilm. This makes it possible to attain nearly 100% utilization of the gas which makes the reaction process more economical than the traditional method of introducing the gas into the water being treated. U.S. Pat. No. 6,387,262 describes an apparatus for safely handling H₂ with minimal risk of explosion. Polyester is permeable to H₂ and O₂, allowing diffusion of the gases across the filaments and into the biofilm.

Third, the use of polyesters allows bioreactors to be produced in essentially any number, from recycled or partially-recycled polyester, with reduced impact to the environment in terms in of e.g., petroleum distillates, new monomer synthesis, reagents and energy for esterification or other synthesis methods, etc. Using an abundant recycled “plastic” material for the manufacture of hollow filaments for bioreactors is an efficient and environmentally-friendly strategy for water treatment.

Fourth, the hollow polyester filaments can be incinerated if they become unsuitable for use, e.g., due to contamination by an undesirable bacterium or other organism. While the hollow polyester filaments can also be disinfected with ozone, peroxide, bleach, etc., it may in some cases be advantageous to incinerate the hollow polyester filaments. Because polyester contains only carbon, hydrogen and oxygen, and does not contain e.g., sulfur and nitrogen, polyester can be incinerated and/or used as fuel, without the production of e.g., toxic sulfur and nitrogen compounds. The use of adhesives, sealants, and related materials low in sulfur and nitrogen for potting the hollow polyester filaments further reduces the amount of toxic waste products produced by incineration.

Exemplary bioreactors and systems will now be described. Reference is made to the Examples below that provide additional details on the modules, bioreactors, and systems.

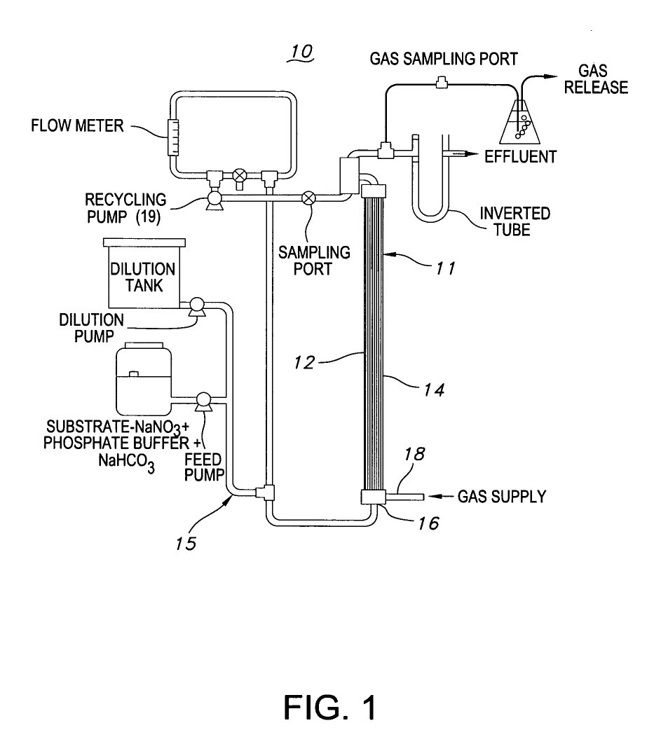

FIG. 1 illustrates an exemplary system 10 comprising a bioreactor 11 comprised of a plurality of hollow filaments 12 housed in a tube enclosure 14. Water to be treated is supplied to the bioreactor via a pipe network, generally designated at 15, that supplies water via a feed pump in communication with water inlet port 16. A controllable amount of gas is introduced into the lumen of the hollow filaments 12 via gas inlet 18. System 10 can optionally include a dilution tank and corresponding pump, to control the concentration of contaminants in the feed to the bioreactor. A recycle pump 19 can be used to improve the mass transfer of the contaminants into the biofilm. Sampling ports and gas release ports are provided as needed. Additionally or optionally, feed water entering bioreactor 11 and contacting the plurality of hollow filaments 12 may be filtered or pretreated using any number of water treatment methods. The direction of water flow is, in this embodiment, around the outside of the filaments 12.

The apparatus may also include one or more side streams for premixing gas, substrates, nutrients, etc. in a volume of water prior to introducing such reagent into the bioreactor. For example, suitable ports, valves, and/or lines for introducing carbon dioxide or an acid, such as sulfuric acid, to the feed water can be included as an approach for adjusting the pH of the feed water.

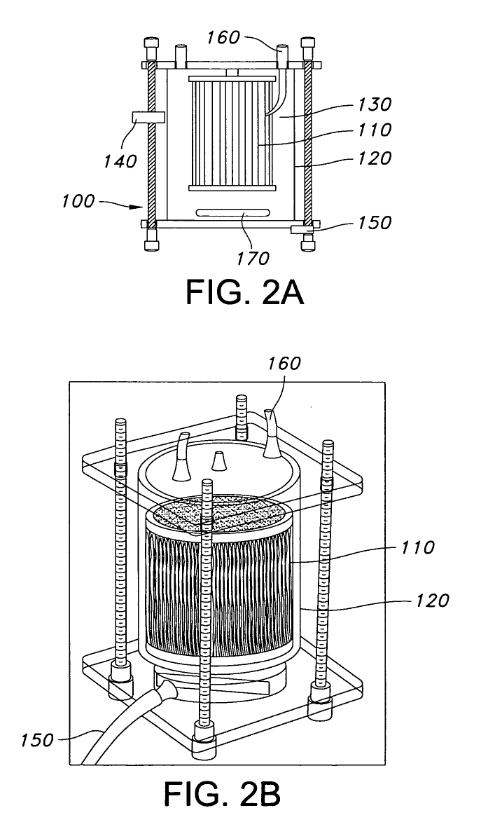

FIG. 2A illustrates an embodiment of a bioreactor, for use in a system for water treatment. Bioreactor apparatus 100 is comprised of a plurality of hollow filaments 110 contained within a vessel 120, which defines a chamber 130 for containing water to be treated. The bioreactor includes an influent port 140 for introducing water to be treated into the vessel, an effluent port 150 for releasing treated water, and a gas supply port 160 for supplying gas to the lumen of the hollow filaments 110. The bioreactor may include a stirring or mixing means, as exemplified by a stir bar 170, in the bioreactor chamber 130. Other mixing means include propellors, gas bubbles, vortexing, and the like. A photograph of a prototype apparatus is shown in FIG. 2B, where the hollow filaments 110, vessel 120, effluent port 150, and gas supply port 160 are indicated.

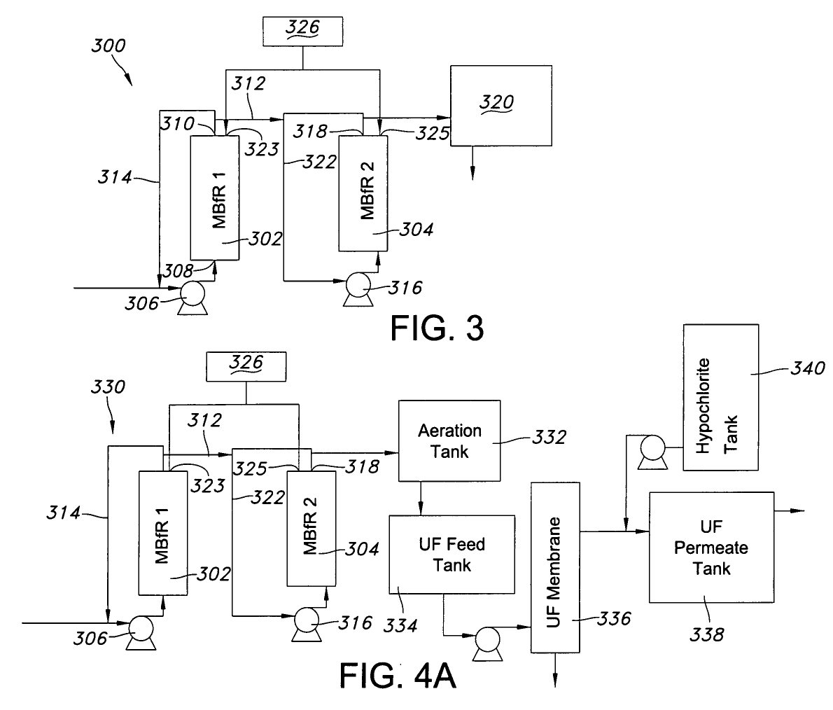

FIG. 3 is a schematic of another exemplary water treatment system 300. System 300 is comprised of a first membrane biofilm reactor (MBfR) 302 and a second MBfR 304, reactors 302, 304 placed in series in the system. Water enters the first bioreactor 302 by an inlet pump 306 at a feed water inlet port 308 of bioreactor 302. Water exits the first bioreactor at exit port 310, whereafter suitable valving is present to direct a portion 312 of the partially treated water into second MBfR 304 or into a recycle line 314 for a second or recycle pass through the first MBfR. Portion 312 of the partially treated water is directed into the second MBfR 304 via pump 316, where it passes through the bioreactor and exits at port 318. Suitable valving is included to direct all or a portion of the water that exits the second MBfR into unit 320, for further processing or into distribution, or into recycle line 322 for another pass through the second MBfR. Bioreactors 302, 304 comprise a plurality of hollow filaments having an external surface and an interior lumen. A gas inlet port in each bioreactor, such as ports 323, 325, are in fluid communication with one or more gas sources, such as source 326, for introducing gas into the lumen of the filaments. The bioreactors in system 300 comprise, in a preferred embodiment, polyester hollow filaments, such as those described above, and a biofilm, as will be described below.

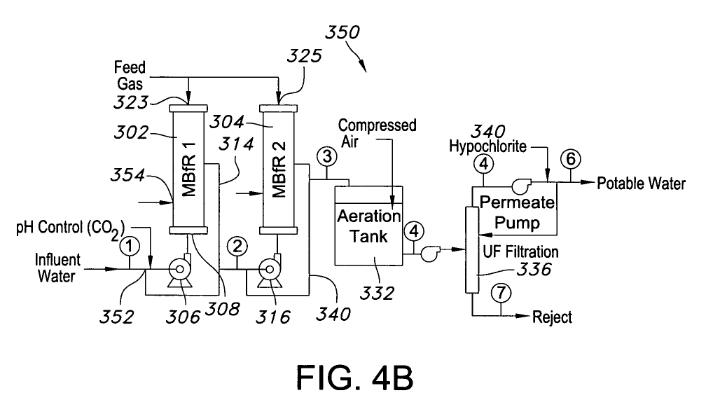

FIG. 4A is a schematic of an exemplary water treatment system 330 for use in municipal water treatment, where like elements with respect to FIG. 3 have like numerical identifiers. In a municipal water treatment system, partially treated or treated effluent water exiting the last MBfR unit, which in this exemplary is the second MBfR unit 304, is directed into an aeration tank 332, and then into an ultrafiltration feed tank 334. Water exits tank 334 and is passed over an ultrafiltration membrane 336 and into a permeate tank 338. A disinfectant, such as hypochlorite held in tank 340, is introduced into the line flowing into the permeate tank 338. It will be appreciated that system 330 can include any combination of the further processing units 332, 334, 336, 338, 340. It will also be appreciated that systems 300 and 330 can include 1, 2, 3, 4, 5, 6, 7, 8, 9, 10, 11, 12, 15, 18, 21, or more MBfR units.

FIG. 4B is a schematic of yet another exemplary water treatment system 350, where like elements with respect to FIG. 3 have like numerical identifiers. In this system, influent water is admixed with a component to adjust the pH of the water introduced into bioreactor 302, such as pH control point 352 where, in this embodiment, carbon dioxide is introduced into the influent line. Bioreactors 302, 304 also comprise a port, such as port 354 on bioreactor 302, for introducing an inert gas, such as nitrogen, into the water treatment side of the hollow filaments. Supply of an inert gas continuously or periodically can be used to providing mixing, fluffing, or aeration of the biofilm that accumulates on the exterior surfaces of the filaments in the bioreactors. Gas introduced on the external surface side of the filaments can also be used to reduce biomass or remove a portion of the biomass. Sample ports, such as the ports numbered 1-7 in system 350, provide access to the water throughout the system for sampling and analysis.

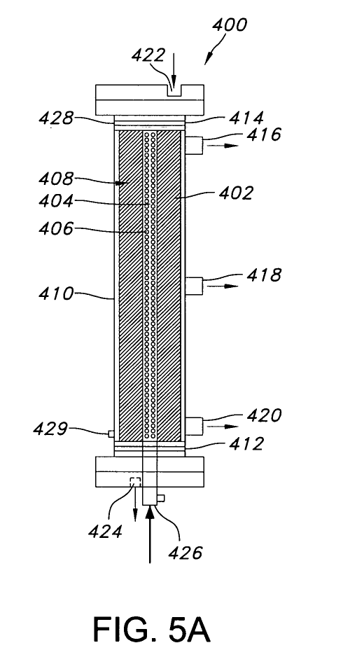

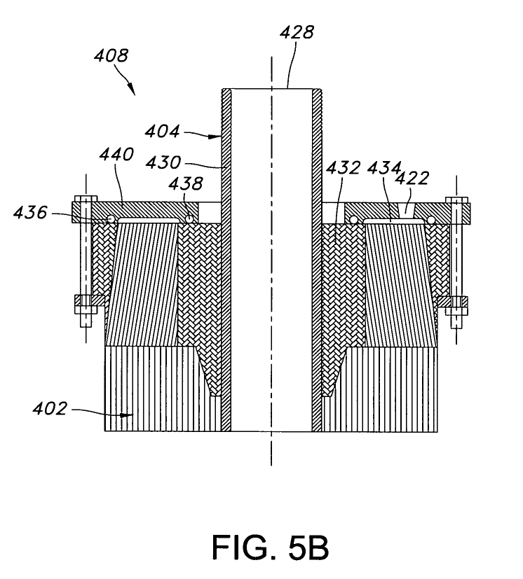

The bioreactors (MBfR5) included in systems like those described in FIG. 3 and FIGS. 4A-4B can take a variety of configurations, and a preferred exemplary configuration is shown in FIGS. 5A-5B. FIG. 5A shows a bioreactor 400 comprised of a plurality of hollow filaments, visible in FIG. 5A as shaded region 402. The plurality of hollow filaments 402 is readily visible in FIG. 5B, discussed below. The hollow filaments extend along, or are wrapped or wound about, a center hollow tube 404 that comprises a plurality of perforations, such as perforation 406. The hollow filaments are secured to the center tube by means known in the art, and described below with respect to FIG. 5B. Together, the hollow filaments, the center tube, and any materials or layers supporting or separating the hollow filaments form a hollow fiber membrane module 408 that is used in or as a bioreactor. Typically, module 408 is inserted into a housing or casing 410, and one or more gaskets or o-rings, such as O-rings 412, 414, provide a fluid tight seal of the module to the casing at either end. The casing includes one or more outlet ports, such as ports 416, 418, 420, for release of treated water from the bioreactor. An inlet port 422 is provided for introducing gas into the lumen of the hollow filaments. At the opposing end of the bioreactor, a drain valve 424 that is movable between a closed position and an open position is provided. Valve 424 when in a closed position prevents release of gas from the lumens of the hollow filaments, permitting a build-up of pressure inside the filaments. As needed, valve 424 can be moved to its open position, for release of gas or discharge of moisture or components in the gas that do not permeate the hollow filaments. Feed water to be treated is introduced into the bioreactor at one end of hollow center tube 404, such as end 426, and opposing end 428 of the center tube is sealed in a fluid tight fashion. Feed water exits the perforations along the length of the center tube, for contact with the external surfaces of the hollow filaments and a biofilm that is supported thereon. Bioreactor 400 can optionally include a gas inlet port 429 for introducing a gas into the water being treated in the bioreactor. Gas can be introduced for a variety of purposes, including aerating the biomass, reducing the biomass, cleaning the exterior surfaces of the biofilm, pH control, etc.

FIG. 5B is an exploded, cross-sectional view of one end of the exemplary module 408 in the bioreactor of FIG. 5A. Center tube 404 is seen in cross-sectional view, and perforations in tube wall 430 are not visible in this view. Hollow filaments 402 in this embodiment, are preferably in the form of a fabric, and layers of the fabric are separated by a spacer material, such as a thermoplastic netting material (commercially available from, for example, DelStar Technologies, Inc.). The fabric and spacer material are secured to the center tube by a sealant, adhesive, expoy or resin, to form a tubesheet 432. The hollow filaments in the fabric extend through the tubesheet and are in fluid communication with a channel 434 which is accessible to the external environment via port 422. Gas, such as hydrogen or oxygen, introduced via port 422 flows into channel 434 and into the hollow interior of the filaments in the fabric wound around the center tube. O-rings 436, 438 seal a cap member 440 to the tubesheet and center tube. Center tube 404 is closed or sealed at the end 428 shown in the figure, and can be threaded at one or both ends, if desired or needed to adapt the module to the system.

In use, gas is directed through port 422 and into the plurality of filaments, which can be any of the materials described herein, and in a preferred embodiment are a polyester. Depending on whether the filaments are in the form of a fabric, individual fibers, twisted yarns, or braids, the filaments may be secured to the center tube or the tube sheet individually or together at one or both ends. In some embodiments, one end of the filaments may be unsecured to the center tube or to a tube sheet, allowing the fibers to move freely and independently at one end. The opposite end of the filaments may be sealed to prevent the escape of gas or open to allow excess gas to flow through.

Gas is directed into the individual filaments, where it permeates across the filaments and becomes available to bacteria growing on the outside surfaces of the hollow filaments (i.e., the biomass or biofilm). In this manner, gas is delivered almost directly to the biomass. Depending on the material and construction of the filaments and the gas pressure, the flux of gas through the filaments will vary, as will be appreciated by a skilled artisan. The choice of polymer, the thickness of the wall of each filament, the operating gas pressure, all influence the permeability of a given gas across the filaments, and its availability to a biofilm growing on the external surface of the filaments. In a preferred embodiment, the filaments are polyester filaments that are non-porous such that the permeability of hydrogen is a function of the particular polymer, gas pressure and wall thickness rather than a function of the physical porosity. In one embodiment, the hydrogen permeability of the filaments is in the range of 0.1-0.8×10⁻¹³ mL-cm/cm²-sec-Pa. In another embodiment, the oxygen permeability of the filaments is in the range of 0.015-0.04×10⁻¹³ mL-cm/cm²-sec-Pa.

Modules used in a bioreactor can also take the form of a collection or plurality of hollow filaments, polyester or other material, arranged into groups and held within a tube, such as tube 14 in FIG. 1. The tube may comprise a plurality of such groups of filaments. One or more tubes or modules of filaments may be connected in series or in parallel to form a reactor. It will be appreciated that bioreactors in parallel are also contemplated, and it will also be appreciated that a system with two or more reactors in series in combination with two or more reactors in parallel are contemplated. For example, a system with multiple reactors can be configured such that a first set of two or more reactors operate in parallel and feed into a second set two or more reactors operating in parallel, which can then feed into a third set of two or more reactors operating in parallel. In some embodiments, the center tube is not present in the module; rather, the hollow filaments are exposed directly to waste water, sand, mud, silt, etc. in a pond, reservoir, contaminated site, or other body.

For most embodiments, the lengths of the hollow filaments are not critical to the function of the bioreactor. However, since the total surface area of the plurality of filaments is proportional to the amount of biomass that can be supported, and ultimately the amount of contaminated water that can be treated, long filaments are preferred for high throughput applications. Such filaments may be several centimeters to several meters in length, depending on design constraints, such as the depth of a reservoir or pond, the length of tube for containing the filaments (if present), the type of gas, the type of oxidized contaminants, and the levels of oxidized and/or reduced contaminants.

In some embodiments, the polyester hollow filaments are provided in a tow of hollow filaments, for example, fine filaments having an outside diameter (OD) of 100 μm or less. The tow of filaments may be potted with minimal reduction in effective surface area. The tows may also be made into open fabrics to facilitate potting, while leaving significant portions of the filaments as tows. Organization of filaments into tows is described, e.g., in U.S. Pat. No. 7,175,763.

In some embodiments, a header directs gas to one (or more often a plurality) of filaments in a reactor in a vertical configuration with the header at the bottom and the hollow filaments floating upwards. In some embodiments, the header is at the top and the hollow filaments oriented downward. In other embodiments, the hollow filaments are oriented outward from the header in the form of a coil, loop, grid, etc. The hollow polyester filaments may be, e.g., at least 50, at least 100, at least 300, at least 500, at least 700, at least 1,000, at least 1,500, and even at least 2,000 μm. Heavier filaments are well suited for coils and loops.

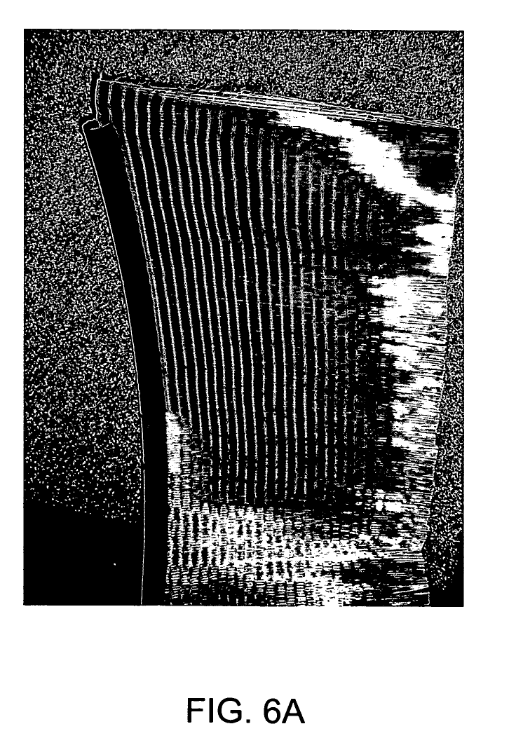

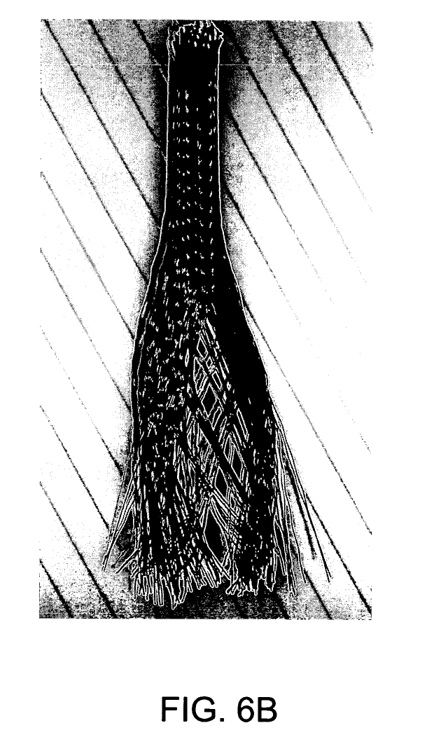

In other embodiments, modules are made from woven, knitted, stitched, braided or other types of fabrics containing a large number of hollow filaments. FIGS. 6A-6B are illustrations of a fabric comprising polyester hollow filaments (FIG. 6A), and of a braid of polyester hollow filaments (FIG. 6B). The fabric may be made with the polyester hollow filaments alone, or in combination with other fibers, particularly inert fibers, that are worked together to form a fabric in a weaving process. In a preferred embodiment the fabric is comprised solely of a polyester material, and filaments in the wart and weft directions are of the same polyester material. Filaments having a diameter of 20 μm or less, or even 10 μm or less, may be used. The fabric imputes strength to the fine filaments to permit biofilm growth on with minimal filament breakage. Modules may be made from fabric sheets with very high packing density to permit good velocities of contaminated water across the exterior surfaces of the hollow filaments without the need to recirculate the water being treated. Examples of such modules are described in U.S. Pat. No. 7,169,295.

In some embodiments, hollow fibers that are melt-spun to have a partially dense, asymmetric, variable porosity, or homogenous wall that does not permit water flow but has gas permeability. Increased permeability results from thermal or physical treatment of the filaments after leaving the spinneret. Examples of this method are described in U.S. Pub. No. 2006-0037896.

In other embodiments, hollow polyester filaments are arranged in a parallel orientation in a sheet or plate having a bottom surface and a top surface. The filament ends at the bottom end are typically open, and the open ends may be potted. Gasses are introduced into the hollow filaments via the potted bottom surface. The filament ends at the top surface may open or closed. Examples of such sheets or plates can be found, e.g., in U.S. Pat. No. 7,140,495.

### IV. Bioreactors

The bioreactors described herein are comprised of a plurality of hollow filaments that provide a support for a biomass or biofilm formed of a bacteria, such as hydrogen-oxidizing bacteria, heterotrophic and/or autotrophic denitrifying bacteria, and/or perchlorate-reducing bacteria, etc. These bacteria for use in forming the biofilm are now described.

A. Denitrifying Bacteria

Denitrifying bacteria include two major groups of aerobic, chemolithoautotrophic bacteria. Ammonia-oxidizing bacteria oxidize ammonia to nitrite, and nitrite-oxidizing bacteria (NOB) oxidize nitrite to nitrate. Collectively, such bacteria are known as nitrifying bacteria or nitrifiers. The first process is performed by a number of facultative anaerobes commonly found in soil. The second process, sometimes referred to as “true” denitrification, is performed by a more select group of bacteria exemplified by Paracoccus denitrificans, Alcaligenes eutrophus, Alcaligenes paradoxus, Pseudomonas pseudoflava, Vibrio dechloraticans Cuznesove B-1168, Acinetobacter thermotoleranticus, Ideonella dechloratans, GR-1 (a strain identified to belong to the β-Proteobacteria, Paracoccus denitrificans, Wolinella succinogenes, and Ralstonia eutropha. Pseudomonas pseudoflava, Alcaligenes eutrophus, Alcaligenes paradoxus, Paracoccus denitrificans, and Ralstonia eutropha can all use hydrogen gas as an electron donor. Ralstonia eutropha is a preferred bacteria available from the American Type Culture Collection (ATCC; Manassas, Va., USA) as collection number 17697.

B. Perchlorate-Reducing Bacteria

Perchlorate-reducing bacteria are generally facultative anaerobes or microaerobes. The bacteria use, for example, acetate, propionate, isobutyrate, butyrate, valerate, malate, fumerate, lactate, and hydrogen as electron donors.

Most perchlorate-reducing bacteria are Proteobacteria. Dechloromonas, Dechlorosoma, and strain GR-1 are β-Proteobacteria, while Azospirillum is an α-Proteobacteria. Strains of Dechloromonas and Dechlorosoma can use lactate as an electron donor, and strains of Dechlorosoma can use ethanol as an electron donor. Some autotrophic Dechloromonas strain use hydrogen as an electron donor. Such strains are particularly useful for use with the present apparatus, systems and methods.

C. Hydrogen-Oxidizing Bacteria

Hydrogen-oxidizing bacteria include both hydrogen-oxidizing, autotrophic bacteria and bacteria able to utilize organic carbon and other energy sources in addition to hydrogen. Hydrogen-oxidizing bacteria are preferred in some embodiments of the present apparatus, systems, and methods. In the presence of oxidized contaminants, such bacteria reduce an oxidized form of a primary electron acceptor in a sufficient amount to sustain a viable, steady-state biomass within the aqueous water-treatment system.

Examples of hydrogen-oxidizing bacteria include but are not limited to Pseudomonas pseudoflava, Alcaligenes eutrophus, Alcaligenes paradoxus, Paracoccus denitrificans, and Ralstonia eutropha.

It will be appreciated that one or more of the above-identified bacteria can be inoculated into a reactor comprising hollow filaments, for formation of a biofilm on the external surfaces of the hollow polyester filaments. It will also be appreciated that one or more of the above-identified bacteria may exist endogenously in the water to be decontaminated, so that inoculation of the reactor is not required. Reliance on endogenous bacteria has the advantage that the bacteria present in the waste water are already adapted to the available nutrients and; therefore, are already capable of metabolizing oxidized contaminant in the waste water, albeit at low efficiency. Bacteria that require similar growth conditions (e.g., feeding with the same gas) may be grown in the same biofilm, while bacteria with different growth requirements (e.g., H₂ or O₂) are typically grown in different bioreactors, for example, a nitrification reactor and a denitrification reactor.

E. Carbon Sources

Where bacteria require a carbon source for growth (e.g., in the case of heterotrophic denitrification), one or more carbon sources can be added to waste water. Example of readily available and inexpensive carbon sources include but are not limited to methanol and acetate; however, carbohydrates, amino acids, lipids, and other carbon sources can be used by bacteria. Where the water to be treated contains a significant amount of trace carbon compounds, the supplementation of the waste water with an additional carbon source may be unnecessary for growth.

F. Mixed Gases/Electron Donors

In some cases, a plurality of gases is introduced into the lumen of the hollow filaments. In some embodiments, one or more electron donor gases are combined with other gases, such as carbon dioxide. Alternatively, other gases can be added separately.

In other embodiments, the mixed gases are a plurality of electron donor gases. For example, where H₂-oxidizing bacteria are used for denitrification, H₂ can be replaced or supplemented with another electron donor gas such as methane, ethane, propane, or alternatively replaced with an electron acceptor gas such as oxygen.

Where a plurality of gases is used, the aerobic/anoxic conditions can be regulated or optimized by variation of one or more partial pressures of the gases in the plurality. While it is possible to obtain biofilms of nitrifying or denitrifying bacteria in a single bioreactor by feeding oxygen or hydrogen, respectively, several days growth may be required to obtain sufficient biomass to metabolize oxidized contaminants. It is, therefore, generally preferred to form and retain a biofilm of nitrifying bacteria in one bioreactor and form and retain a biofilm of denitrifying bacteria in another bioreactor. A biofilm of chlorate-reducing bacteria may be formed and retained in yet another bioreactor.

### V. Waste Water Contaminants

The present apparatus, systems, and methods can be used to remove a variety of contaminants from water, both oxidized contaminants, such as nitrate (NO₃⁻) and nitrite (NO₂⁻), and reduced nitrogen-containing compounds, such as ammonia (i.e., the ammonium ion; NH₄+), aniline, nitraamines, nitroaromatics, nitrobenzene, and trinitrotoluene.

Additional contaminants include halogen-containing compounds, such as perchlorate (ClO₄⁻), chlorate (ClO₃⁻), chlorite (ClOC₂⁻), methyl chloride, methylene chloride, chloroform, dichloromethane, dichloroethane, trichloroethylene, vinyl chloride, tetrachloromethane, chlorobenzene, dinitrotoluene, dioxane, perchloroethylene, trichloroethane, trichlorotoluene, chlorinated ethenes, chlorinated ethanes, bromoform and bromate (BrO₃⁻). Yet further contaminants include sulfur-containing compounds, such as hydrogen sulfide and carbon disulphide, which can be oxidized with oxygen to sulfate.

Other oxidized contaminants and reduced contaminants include alcohols and phenols, such as methanol, ethanol, butanol, 1-propanol, 2-propanol, tertiary butyl alcohol, n-butyl alcohol, glycols, phenols; ketones, e.g., acetone, methylethyl ketone; aromatic solvents, e.g, tetrahydrofuran, toluene, benzene, naphthalene; assorted other hydrocarbons, solvents, and pesticides; as well as selenate (SeO₄²⁻), selenite (HSeO₃⁻), chromate (CrO₄²⁻), arsenate (H₂AsO₄⁻).

The present apparatus, systems, and methods apply to any one or more of these contaminants and/or apply to all the listed contaminant with the exception of any one or more of the listed contaminants. In one embodiment, nitrate is removed to a concentration of less than about 10 mg-N/L, and nitrite is removed to a concentration of less than about 1 mg-N/L.

### V. Sequential, Simultaneous, and Coupled Processing

The present apparatus, systems, and methods comprising hollow filaments can be applied to removal of oxidized contaminants from industrial waste water, contaminated surface water or ground water, or drinking water. The apparatus may also be used to remove oxidized contaminants or reduced contaminants from brines and waste waters (municipal, agricultural, or industrial). The apparatus may be installed within an existing treatment system, e.g., before or after the filtration process or disinfection processes, installed at the end of an existing treatment system to further process the waste water, or used in place of an existing water treatment system. The apparatus may also be used to decontaminate ground water, soil, mud, silt, etc., in situ.

As noted above, treatment of water can be performed sequentially, i.e., using the same plurality hollow polyester filaments with different bacterial biofilms, or using a separate plurality of hollow polyester filaments for different bacterial biofilms. For example, the aerobic/anoxic conditions in a plurality hollow polyester filaments can be changed based selection of gas introduced into the filaments. In other embodiments, separate bioreactors are used for, e.g., nitrifying and denitrifying bacteria, and, optionally, perchlorate-reducing bacteria.

For example, in one embodiment for total nitrogen removal from water, reduced nitrogen contaminants, such as ammonia (or ammonium) and nitrite, are first oxidized to nitrate (or to nitrite, and then to nitrate) in an aerobic reactor in which oxygen is fed into hollow polyester microfilaments to support the growth of nitrifying bacteria for formation of a biofilm. The water is then transferred to an anoxic reactor for the further reduction of nitrate to nitrogen gas by hydrogen-oxidizing bacteria, maintained as a biofilm on the exterior filament surfaces by feeding hydrogen gas into the hollow polyester filaments of the anoxic reactor.

In this embodiment, the processing of a given sample/volume of waste water is sequential with respect to the aerobic reactor and then the anoxic reactor. However, processing of one sample/volume in the aerobic reactor, and another sample/volume in the anoxic reactor, can occur simultaneously. Moreover, since the amount of acidic byproducts produced during nitrification is about equal to the amount of basic byproducts produced during denitrification, the reactions can be coupled by recycle flow between the reactors.

For the removal of perchlorate and chlorate, perchlorate-reducing bacteria are used in a bioreactor arranged upstream or downstream of a nitrifying and/or denitrifying reactor. As above, the processing of a given sample/volume of waste water is sequential with respect to perchlorate removal and nitrogen contaminant removal. However, processing of one sample/volume in the perchlorate reactor and another sample/volume in nitrifying and/or denitrifying reactor may occur simultaneously.

These and other applications and implementations will be apparent to artisans.

### VI. EXAMPLES

The following examples are illustrative of the apparatus, systems, and methods described herein and are not intended to be limiting to the scope of the subject matter.

### Example 1

### Module with Polyester Hollow Filaments

A module for use as a bioreactor in a system was prepared as follows. A bundle comprised of 240 polyester hollow filaments, each 43 cm in length and having a 130 μm outer diameter, was sealed using a suitable epoxy into a plastic casing member, according to techniques known in the art, to form a module. The total membrane area was 0.0423 m². One end of the filaments extended through the epoxy such that the internal bore of each fiber was open and in fluid communication with an inlet port on a cap member placed at the end of the casing. The opposing end of each filament was sealed into the epoxy. Flange members were fitted onto the casing, for coupling the module to a water feed source that is introduced onto the shell side, or external surfaces, of the filaments. In use, the inlet port is coupled to a gas source, for introducing gas to the internal bore of the hollow filaments. An outlet port in the module casing was provided to allow decontaminated water to exit the module.

Hydrogen flow of the polyester filaments was tested at operating pressures of 12, 13, 15, 20, 25, 30, 45 psig. The filaments were soaked in water for 24 hours prior to initiating the test. Hydrogen was introduced via the port in the cap member, and the volume of H₂ collected at the module outer surface varied independent of pressures under 25 psig. No formation of bubbles in the external environment was observed when the hydrogen pressure was below 25 psig. However, undesirable bubbles formed on the outer surface of filaments at 30 psig and 45 psig. The hydrogen flow rate was 0.09 mL/hr at 30 psig and 0.22 mL/hr at 45 psig. These flow rates correspond to a hydrogen fluxes of 2.09 mL/m²-hr and 5.2 mL/m²-hr, respectively. Thus, for this module, a recommend operating pressure is about 25 psig, with an increase to 30 psig possible. In use, the reactor can first be operated at a pressure of around 12-13 psig, and after a biofilm is formed on the outside surfaces of the filaments, a higher pressure of up to 25-30 psig can be used.

### Example 2

### Module with Fabric Comprising Polyester Hollow Filaments

A module for use as a bioreactor in a system was prepared as follows. An aluminum tube having an inner diameter of 2 inches (5.1 cm) was prepared by introducing a series of holes along its length. A sheath of fabric comprised of polyester hollow filaments woven to form a fabric like that depicted in FIG. 6A, and a mesh spacer material were wound around the outer circumference of the tube, according to techniques known in the art, to form a spiral wound module. An epoxy seal was placed along the fabric and mesh outer edges subsequent to winding, to provide an epoxy seal at each end of the module. The MBfR module is approximately 41 inches long and five and one-half inches in diameter. The MBfR module contained approximately 26,800 hollow fibers having 200 micron outer diameter (OD) and with 36 inches of active length (after subtracting length covered by the epoxy-potted ends). The MBfR active surface area was therefore 15.4 square meters. The module was as shown in FIG. 5A and was comprised of several components. The primary components were the MBfR casing, the perforated core tube, the hollow membrane fibers and potted tube sheet at each end. On the casing there were three discharge ports and three gas scouring ports (note that in FIG. 5A only one gas scouring port 428 is shown). The three gas scouring ports were distributed equally distant around the case. In the center of the module was the core tube. The core tube was sealed into the tube sheets at each end and the core tube was pluged at its top end forcing water, introduced at the opposing open end of the core tube, to flow radially out through the perforations in the core tube into the fibers. The core tube also had one gas scouring port. At each end of the module was a tube sheet. All of the membrane hollow fibers and the core tube were sealed into the tube sheet. The tube sheet had an O-ring that sealed the MBfR module into a casing, to form a bioreactor (module plus casing). The top of the MBfR reactor was equipped with a hydrogen addition port that allowed hydrogen (or another gas, such as oxygen) to be introduced into the lumen or bore of the hollow fiber membrane. The bottom of the MBfR reactor was equipped a drain valve to allow inerts to be drained from the bore of the fibers.

### Example 3

### Module with Fabric Comprising Polyester Hollow Filaments

A module for use in a bioreactor system was prepared as follows. A PVC tube having an inner diameter of 2 inches (5.1 cm) was prepared by introducing a series of holes along its length. A sheath of fabric comprised of polyester hollow filaments woven to form the fabric, like that depicted in FIG. 6A, and a mesh spacer material were wound around the outer circumference of the tube, according to techniques known in the art, to form a spiral wound module, where the spacer material was adjacent and separating layers of the polyester fabric. An epoxy seal was placed along the fabric and mesh outer edges prior to winding, to provide an epoxy seal at each end of the module. Flange members were fitted into the epoxy, for coupling the module to a water feed source. The end of each module was trimmed, and a stainless steel cap was secured to one end of the module, the cap having a port for fluid communication between the environment of use and the interior of the polyester filaments. The module in combination with the cap and its port define, in this embodiment, a bioreactor. The cap also contained a groove to define a channel through which the port and the polyester filaments communicate (see FIG. 5B). In use, the port is coupled to a hydrogen source, for introducing hydrogen gas to the bore side of the hollow filaments. The opposing end of the module was sealed so that the hollow filaments in the fabric were closed, and an outlet port on the shell side of the case was provided to allow decontaminated water to exit the bioreactor.

### Example 4

### Module with Polyethylene Naphthalate Filaments

A module for use as a bioreactor in a system is prepared as follows. A bundle comprised of 480 hollow filaments of polyethylene naphthalate, each 50 cm in length and having a 160 μm outer diameter, are sealed using a suitable epoxy to form a tube sheet, and then inserted into a plastic casing member, according to techniques known in the art, to form a module. Both ends of the filaments extend through tubesheet, and are open to the casing member. At one end of the casing member is a valve that moves to a closed position to close the filaments on one end. At an opposing end of the casing is an inlet port on a cap member, the inlet port in fluid communication with a gas source. Flange members are fitted onto the casing, for coupling the module to a water feed source that is introduced onto the shell side, or external surfaces, of the filaments. In use, the inlet port is coupled to a gas source, for introducing gas to the internal bore of the hollow filaments. An outlet port in the module casing is provided to allow decontaminated water to exit the module.

### Example 5

### Module with Filaments of Other Materials

Modules are prepared according to Example 4, but with filaments comprised of the following materials: poly(trimethylene terephthalate) (PTT), poly(butylene terephthalate) (PBT), poly(cyclohexylene dimethylene terephthalate) (PCTA), polycarbonate (PC), poly(butylene naphthalate) (PBN), and poly(lactic acid) (PLA). The modules are secured in a casing, for forming a bioreactor.

### Example 6

### Bench Test with Municipal Wastewater

A study was done using a module prepared as described in Example 1, and in a system similar to that depicted in FIG. 3 but with a single module (membrane biofilm reactor), for removal of the contaminants nitrate (NO₃) and nitrite (NO₂) from municipal wastewater. Operating parameters of the system, and the nitrate and nitrate concentrations, are summarized in the table below. Nitrate and nitrate concentrations are measured on a nitrogen only basis using a calorimetric assay commercially available from Hach Company.

| | | | | | | Re-Circulation |
|---|---|---|---|---|---|---|
| | | | | | Feed Rate of | Rate of Water |
| | | | | | Contaminated | through |
| Day of | H₂ Pressure | NO₃—N | NO₂—N | | Water | Bioreactor |
| Operation | (psig) | mg/L | mg/L | pH | (mL/min) | (mL/min) |
| 0 | | 12.3 | 0.04 | 8.3¹ | | |
| Inlet Water | | | | | | |
| Characteristics | | | | | | |
| 1 | 13.5 | 2.3 | 0.086 | 9.12 | 0.25 | 280 |
| 2 | 13.5 | 0 | 0.01 | 9.22 | 0.25 | 280 |
| 3 | 13.5 | 2.2 | 0.077 | 9.13 | 0.55 | 280 |
| 4 | 13.5 | 2.3 | 0.004 | 9.07 | 0.55 | 280 |
| 5 | 13.5 | 6.1 | 0.019 | 8.93 | 0.8 | 280 |
| 6 | 13.5 | 6.9 | 0.1 | 8.86 | 0.8 | 280 |
| 7 | 15.5 | 7.2 | 0.009 | 8.92 | 0.8 | 280 |
| 8 | 15.5 | 6.6 | 0.01 | 8.78 | 0.8 | 280 |
| 9 | 15.5 | 0.2 | 0.008 | 8.82 | 0 | 280 |
| 10 | 15.5 | 6.8 | 0.6 | 8.86 | 0.8 | 280 |
| 11 | 15.5 | 0.3 | 0.05 | 9.16 | 0.8 | 280 |
| 17 | 25 | 0.2 | 0.005 | 8.52 | 0.8 | 280 |
| 18 | 25 | 0.8 | 0.71 | 8.86 | 1.2 | 280 |

¹pH of source water; all other pH values measured at water effluent of module.

The removal of nitrate was rapid, with a reduction of more than 5-fold (12.3 mg/L to 2.3 mg/L) observed after one day of operation. The system was initially run at a hydrogen pressure of about 13 psig, and the hydrogen pressure was increased to about 25 psig on day 17 of the test. On day 8, when the nitrate concentration had plateaued for several days, the system was switched via suitable valving such that the bioreactor was operated in full recirculation mode, and no feed water flowed into the module. An immediate reduction in nitrate concentration was observed, such that at day 11, nitrate concentration was 0.3 mg/L. At the end of day 17 of the study, the feed flow rate was increased to 1.2 mL/min, and at the end of day 18 to 2 mL/min. At a feed rate of 1.2 mL/min, the nitrate removal flux was 450 mg-NO₃/m²-day.

### Example 7

### Field Test with Municipal Wastewater

A study was done using a module prepared as described in Example 2, and in a system similar to that depicted in FIG. 3 but with a single membrane biofilm reactor, for removal of the contaminants nitrate (NO₃) and nitrite (NO₂) from municipal wastewater. The system was placed in the field and, the contaminated wastewater fed into the bioreactor. Hydrogen was introduced into the hollow filaments. Operating parameters of the system, and the nitrate and nitrate concentrations, are summarized in the tables below.

| | | | Water | | | | | |
|---|---|---|---|---|---|---|---|---|
| | | | Feed | Recycle | Pressure, psig | | | |
| no. of days | | Temp | Flow | flow | Module | Module | (Inlet − | |
| in operation | Time | (° F.) | (GPM) | (GPM) | Inlet | Outlet | Outlet) | Hydrogen |
| 1 | 8:15 | 60 | 0.19 | 10.00 | 23 | 14 | 9 | 23 |
| 1 | 15:11 | 70 | 0.29 | 10.00 | 20 | 14 | 6 | 27.5 |
| 2 | 8:55 | 62 | 0.22 | 10.00 | 20 | 14 | 6 | 28.5 |
| 2 | 15:40 | 72 | 0.23 | 10.00 | 20 | 13.5 | 6.5 | 25 |
| 3 | 10:00 | 68 | 0.20 | 10.00 | 20.5 | 14 | 6.5 | 28.5 |
| 3 | 16:00 | 70 | 0.22 | 10.00 | 22 | 15.5 | 6.5 | 29 |
| 4 | 8:40 | 64 | 0.21 | 10.00 | 23 | 16 | 7 | 29 |
| 4 | 16:00 | 72 | 0.21 | 10.00 | 21 | 14 | 7 | 28.5 |
| 6 | 15:10 | 72 | 0.20 | 11.00 | 23 | 15 | 8 | 29 |
| 7 | 9:10 | 64 | 0.21 | 10.00 | 22 | 14 | 8 | 29 |
| 7 | 15:45 | 67 | 0.21 | 10.00 | 21 | 14 | 7 | 29.5 |
| 8 | 9:30 | 60 | 0.21 | 10.00 | 21 | 14 | 7 | 28 |
| 8 | 15:15 | 64 | 0.20 | 10.00 | 21 | 14 | 7 | 28.5 |
| 9 | 8:00 | 54 | 0.21 | 10.00 | 23 | 57 | −34 | 28 |
| 9 | 14:30 | 65 | 0.22 | 10.00 | 22 | 15.5 | 6.5 | 27 |
| 10 | 7:25 | 52 | 0.21 | 10.00 | 24 | frozen | | 29.5 |
| 10 | 14:55 | 58 | 0.23 | 10.00 | 23 | 22 | 1 | 28.5 |
| 11 | 8:45 | 56 | 0.21 | 10.00 | 23.5 | 22 | 1.5 | 28 |
| 11 | 3:10 | 64 | 0.23 | 10.00 | 23 | 22 | 1 | off |
| 12 | 11:25 | 54 | 0.21 | 10.00 | 24 | 21 | 3 | 29 |
| 13 | 10:30 | 60 | 0.20 | 10.00 | 21 | 21 | 0 | 0 |
| 13 | 15:45 | 62 | 0.18 | 10.00 | 22 | 20 | 2 | 26 |
| 14 | 11:00 | 64 | 0.22 | 10.00 | 22.5 | 20 | 2.5 | 31 |
| 14 | no data | 62 | 0.21 | 10.00 | 21 | 20 | 1 | 31 |
| 15 | 9:45 | 57 | 0.21 | 10.00 | 23 | 21 | 2 | 26 |
| 15 | 15:25 | 59 | 0.23 | 10.00 | 21.5 | 20 | 1.5 | 28 |
| 16 | 8:50 | 57 | 0.23 | 10.00 | 22.5 | 20 | 2.5 | 29 |
| 16 | 15:50 | 60 | 0.44 | 10.00 | 20 | 18.5 | 1.5 | 27 |
| 17 | 9:50 | 44 | 0.44 | 10.00 | 21 | 19 | 2 | 27.5 |
| 17 | 15:30 | 45 | 0.41 | 10.00 | 20 | 19 | 1 | 29 |
| 18 | 7:45 | 57 | 0.41 | 10.00 | 22 | 19 | 3 | 30.5 |
| 18 | 15:40 | 52 | 0.33 | 10.00 | 22 | 19.5 | 2.5 | 35 |
| 19 | 10:30 | 56 | 0.30 | 6.00 | 22 | 19 | 3 | 33 |
| 20 | 9:10 | 54 | 0.30 | 6.00 | 23 | 20 | 3 | 34 |
| 21 | 10:20 | 60 | 0.31 | 10.00 | 18 | 18.5 | −0.5 | 30 |
| 21 | 15:25 | 64 | 0.33 | 10.00 | 23 | 19.5 | 3.5 | 30 |
| 22 | 10:27 | 61 | 0.45 | 10.00 | 22 | 22 | 0 | 34 |
| 22 | 15:50 | 61 | 0.56 | 10.00 | 22.5 | 21 | 1.5 | 31 |
| 23 | 8:20 | 56 | 0.58 | 10.00 | 27 | 21 | 6 | 23 off |
| 23 | 16:10 | 56 | 0.57 | 10.00 | 14.5 | 21 | −6.5 | 32/27 |
| 24 | 12:00 | 53 | 0.52 | 10.00 | 27 | 22 | 5 | 30 |
| 24 | 15:50 | 53 | 0.57 | 10.00 | 24 | 19 | 5 | 29 |
| 25 | 8:55 | 52 | 0.57 | 10.00 | 24 | 18.5 | 5.5 | 29 |
| 25 | 16:00 | 52 | 0.43 | 10.00 | 26 | 22 | 4 | 30 |
| 26 | 12:30 | 46 | 0.55 | 10.00 | 25 | 20 | 5 | 30 |
| 27 | 10:00 | 48 | 0.53 | 10.00 | 29 | 21 | 8 | 32 |
| 28 | 9:25 | 49 | 0.55 | 10.00 | 29 | 21 | 8 | 28 |
| 28 | 16:30 | 55 | 0.55 | 10.00 | 30 | 20 | 10 | 30 |
| 29 | 9:10 | 49 | 0.58 | 10.00 | 0 | 28 | | |
| 29 | 15:00 | 52 | 0.56 | 2.00 | 16 | 23 | −7 | 30 |
| 30 | 10:15 | 49 | 0.55 | 2.00 | 30 | 23 | 7 | 29 |
| 30 | 15:35 | 50 | 0.57 | 2.00 | 29.5 | 23 | 6.5 | 29.5 |
| 31 | 10:30 | 50 | 0.57 | 2.00 | 27 | 23.5 | 3.5 | 29 |
| 31 | 16:05 | 50 | 0.56 | 2.00 | 28 | 23 | 5 | 29 |
| 32 | 8:50 | 49 | 0.56 | 2.00 | 29 | 23 | 6 | 30.5 |
| 32 | 15:00 | 55 | 0.39 | 2.00 | 28 | 24 | 4 | 30 |
| 33 | 15:00 | 56 | 0.40 | 2.00 | 30 | 24 | 6 | 30 |
| 34 | 15:25 | 50 | 0.44 | 2.00 | 26 | 23 | 3 | 29 |
| 35 | 8:40 | 51 | 0.42 | 2.00 | 30 | 23 | 7 | 30 |
| 35 | 15:20 | 52 | 0.40 | 2.00 | 27.5 | 23 | 4.5 | 27 |
| 36 | 9:00 | 53 | 0.42 | 2.00 | 30 | 23 | 7 | 30 |
| 36 | 16:00 | 54 | 0.30 | 2.00 | 30 | 23 | 7 | 29 |
| 37 | 9:16 | 54 | 0.31 | 2.00 | 30 | 23 | 7 | 30 |
| 37 | 15:00 | 54 | 0.32 | 2.00 | 25 | 22 | 3 | 28.5 |
| 38 | 11:10 | 49 | 0.30 | 2.00 | 29 | 23 | 6 | 30 |
| 38 | 16:25 | 49 | 0.32 | 2.00 | 26 | 23 | 3 | 31 |
| 39 | 7:50 | 45 | 0.31 | 2.00 | — | — | — | 31 |
| 39 | 15:30 | 51 | 0.28 | 2.00 | 26 | 22 | 4 | 29 |
| 40 | 8:30 | 50 | 0.31 | 2.00 | 22 | 19 | 3 | 28 |
| 41 | 9:15 | 53 | 0.30 | 2.00 | 20.5 | 18 | 2.5 | 27 |
| 43 | 15:00 | 47 | 0.25 | 2.00 | 26 | 22.5 | 3.5 | 31 |
| 46 | 12:30 | 59 | 0.20 | 10.00 | 31 | 11 | 20 | 31 |
| 47 | 15:15 | 58 | 0.47 | 10.00 | 30 | 11 | 19 | 29 |
| 48 | 8:20 | 50 | 0.44 | 10.00 | 34 | 12 | 22 | 33 |
| 49 | 15:30 | 54 | 0.53 | 10.00 | 31.5 | 12 | 19.5 | 31 |
| 50 | 16:10 | 46 | 0.54 | 10.00 | 32 | 12 | 20 | 50 |

Summary of Data for System Operated at Recycle Rate of 10 GMP

| | Hydraulic | Water | Nitrate | | Nitrite- | Nitrite- | Nitrate | | |
|---|---|---|---|---|---|---|---|---|---|
| | Residence | Feed | Concentration* | | IN* | OUT* | loading to | Nitrate | % |
| Day in | Time | Flow | mg-N/L | | mg- | mg- | System | Flux | Nitrate |
| operation | (min) | (GPM) | Feed | Effluent | N/L | N/L | mg-N/m² · d | mg-N/m² · d | removal |
| 11 | 50.0 | 0.21 | 18.10 | 0.76 | 0.26 | 0.67 | 1183.8 | 1134.1 | 95.8 |
| 14 | 47.7 | 0.22 | 12.30 | 0.11 | 0.11 | 0.10 | 842.8 | 835.3 | 99.1 |
| 16 | 45.7 | 0.23 | 16.50 | 1.96 | 0.14 | 1.70 | 1182.0 | 1041.6 | 88.1 |
| 18 | 23.9 | 0.44 | 12.80 | 1.84 | 0.23 | 2.87 | 1754.1 | 1501.9 | 85.6 |
| 23 | 18.1 | 0.58 | 16.10 | 12.60 | 0.12 | 0.35 | 2908.3 | 632.2 | 21.7 |
| 25 | 18.4 | 0.57 | 16.80 | 14.60 | 0.11 | 0.84 | 2982.5 | 390.6 | 13.1 |
| 28 | 19.1 | 0.55 | 20.00 | 15.2 | 0.1 | 1.1 | 3426.0 | 822.2 | 24.0 |

*Nitrate and nitrite concentrations shown on a nitrogen-only basis.

Summary of Data for System Operated at Recycle Rate of 2 GMP

| | Hydraulic | Water | Nitrate | | Nitrite- | Nitrite- | Nitrate | | |
|---|---|---|---|---|---|---|---|---|---|
| | Residence | Feed | Concentration* | | IN* | OUT* | loading to | Nitrate | % |
| Day in | Time | Flow | mg-N/L | | mg- | mg- | System* | Flux | Nitrate |
| operation | (min) | (GPM) | Feed | Effluent | N/L | N/L | mg-N/m² · d | mg-N/m² · d | removal |
| 30 | 19.1 | 0.55 | 20.2 | 16.0 | 0.1 | 0.5 | 3460.2 | 719.5 | 20.8 |
| 32 | 33.9 | 0.31 | 15.8 | 3.9 | 0.1 | 1.0 | 1525.5 | 1147.0 | 75.2 |
| 35 | 25.0 | 0.42 | 14.0 | 5.9 | 0.3 | 2.9 | 1831.3 | 1064.8 | 58.1 |
| 37 | 33.9 | 0.31 | 16.6 | 4.7 | 0.2 | 3.7 | 1602.7 | 1153.8 | 72.0 |
| 39 | 33.9 | 0.31 | 19.3 | 8.7 | 0.1 | 6.6 | 1863.4 | 1027.3 | 55.1 |
| 41 | 35.0 | 0.30 | 16.8 | 7.2 | 0.3 | 3.0 | 1569.7 | 896.0 | 57.1 |

*Nitrate and nitrite concentrations shown on a nitrogen-only basis.

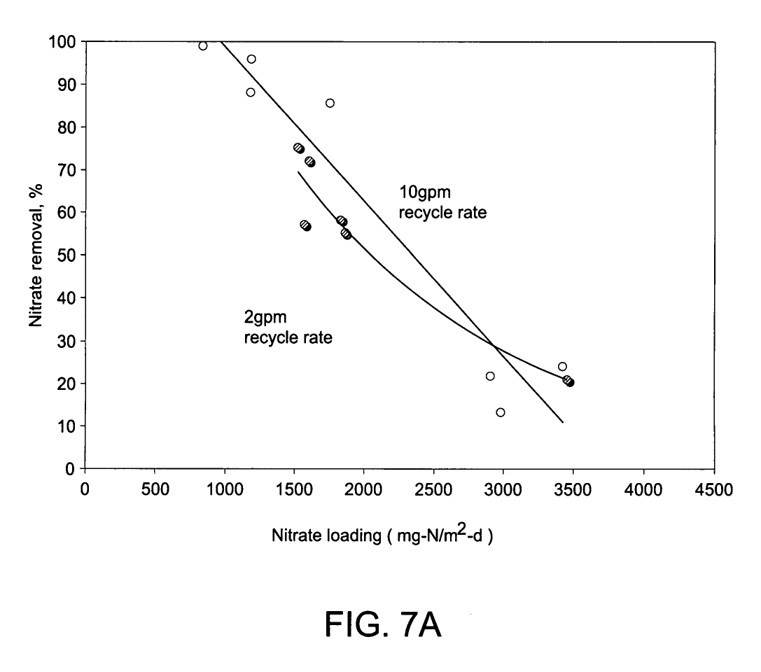

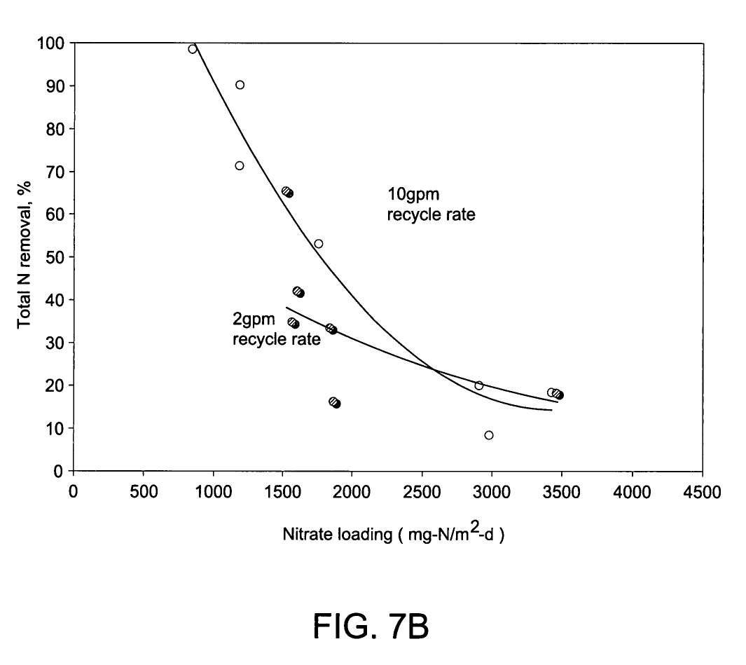

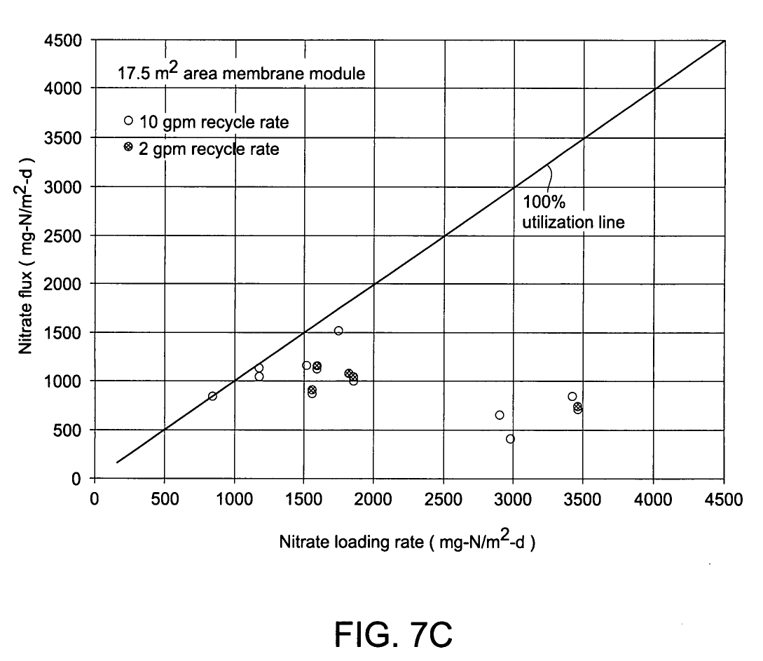

The removal of nitrate and nitrite as a function of nitrate loading, at recycle rates of 10 gallons per minute (0.63 L/s, open circles) or 2 gallons per minute (0.13 L/s, closed circles) is shown in FIGS. 7A-7B. Specifically, FIG. 7A is a graph of nitrate removal, in percent, as a function of nitrate loading (on a nitrogen-only basis), mg-N/m²-day, during treatment of wastewater in a system comprising a single bioreactor having one module with polyester hollow filaments, the bioreactor operated with a recycle rate of 10 gallons per minute (0.63 L/s, open circles) or 2 gallons per minute (0.13 L/s, closed circles). FIG. 7B is a graph of total nitrogen removal, in percent, as a function of nitrate loading rate, in mg-N/m²-day, in a system for treatment of wastewater, the system comprising a single bioreactor module with polyester hollow filaments, the bioreactor operated with a recycle rate of 10 gallons per minute (0.63 L/s, open circles) or 2 gallons per minute (0.13 L/s, closed circles).

FIG. 7C is a graph of nitrate flux, in mg-N/m²-day, as a function of nitrate loading rate, in mg-N/m²-day, in a system for treatment of wastewater, the system comprising a single bioreactor module with polyester hollow filaments, the bioreactor operated with recycle rates of 10 gallons per minute (0.63 L/s, open circles) or 2 gallons per minute (0.13 L/s, closed circles). The flux of nitrate is calculated from the difference of nitrate concentrations in and out of the system divided by the membrane area per day. The solid line labeled “100% utilization line” identifies where for any given loading of nitrate into the system, the points at which the flux of nitrate (i.e., removal of nitrate) from the system is essentially complete. As seen, complete removal of nitrate was achieved at nitrate loadings of up to about 1200 mg-N/m²-day using this system.

While various embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

It is claimed:

1. An apparatus, comprising: a plurality of hollow filaments, each hollow filament comprised of a polyester.

2. The apparatus of claim 1, wherein each hollow filament in the plurality of hollow filaments is comprised of a polyester selected from the group consisting of poly(ethylene terephthalate) (PET), poly(trimethylene terephthalate) (PTT), poly(butylene terephthalate) (PBT), poly(ethylene naphthalate) (PEN), poly(cyclohexylene dimethylene terephthalate) (PCTA), polycarbonate (PC), poly(butylene naphthalate) (PBN), and poly(lactic acid) (PLA).

3. The apparatus of claim 1, wherein each hollow filament in the plurality of hollow filaments is comprised of a polyester having a carboxylate ester of the form R₁—C(═O)OR₂ in its monomeric repeat unit.

4. The apparatus of claim 3, wherein the polyester is poly(ethylene terephthalate) (PET).

5. The apparatus of claim 1, wherein the plurality of hollow filaments are secured at one end to a tube sheet.

6. The apparatus of claim 1, wherein the plurality of hollow filaments form a fabric.

7. The apparatus of claim 6, wherein the plurality of hollow filaments are sealed at one end and open at an opposite end, and the fabric is secured to a center tube.

8. The apparatus of claim 7, wherein the center tube is perforated.

9. The apparatus of claim 6, further comprising a spacer material adjacent the fabric.

10. An apparatus, comprising:
a plurality of hollow filaments, each hollow filament comprised of a material selected from the group consisting of a polyester, a polyamide, a halogenated polymer, a non-halogenated polyolefin, and a sulfur-containing polymer.

11. The apparatus of claim 10, wherein the plurality of hollow filaments forms a fabric.

12. The apparatus of claim 11, wherein the plurality of hollow filaments are sealed at one end and open at an opposite end, and the fabric is secured to a center tube.

13. The apparatus of claim 11, further comprising a spacer material adjacent the fabric.

14. The apparatus of claim 11, further comprising a drain valve.

15. The apparatus of claim 11, wherein further comprising a casing having a central cavity, wherein said plurality of hollow filaments are inserted into the casing.

16. The apparatus of claim 15, further comprising a port on the casing, the port in fluid communication with an external surface of the plurality of hollow filaments and an environment of use.

17. A bioreactor, comprising
an apparatus according to claim 1, and
a port for introducing a gas into said apparatus.

18. The bioreactor of claim 17, wherein each filament in the plurality of hollow filaments has an exterior surface and an interior surface defining a hollow interior and the port is in fluid communication with the hollow interior of the plurality of hollow filaments.

19. The bioreactor of claim 17, wherein a gas selected from hydrogen and oxygen is introduced into the apparatus.

20. The bioreactor of claim 17, situated downstream a denitrification process supplied with a carbon source.

21. The bioreactor of claim 18, wherein the carbon source is selected from acetate, propionate, isobutyrate, butyrate, valerate, malate, fumerate, lactate, chlorate, methanol, catechol, glycerol, and citrate.

22. A method of removing a contaminant from wastewater, comprising:
providing a plurality of hollow filaments, the hollow filaments each having an exterior surface and an interior surface defining a hollow interior, and being comprised of a polyester;
providing a gas to the hollow interiors of the hollow filaments;
and contacting wastewater with the exterior surface of the hollow filaments, wherein a biofilm on the exterior surfaces of the plurality of hollow filaments removes a contaminant in the wastewater.

23. The method of claim 22, wherein said gas is selected from the group consisting of hydrogen, oxygen, and nitrogen.

24. The method of claim 22, wherein said plurality of hollow filaments are contained in a casing that defines a cavity for containing said plurality of hollow filaments, and said method further comprises introducing a gas to said cavity.

25. The method of claim 22, further comprising adding a gas to said wastewater.

26. The method of claim 23, wherein said gas is carbon dioxide.

27. The method of claim 22, wherein said polyester is selected from the group consisting of poly(ethylene terephthalate) (PET), poly(trimethylene terephthalate) (PTT), poly(butylene terephthalate) (PBT), poly(ethylene naphthalate) (PEN), poly(cyclohexylene dimethylene terephthalate) (PCTA), polycarbonate (PC), poly(butylene naphthalate) (PBN), and poly(lactic acid) (PLA).
